(19) United States
(12) Patent Application Publication
(10) Pub. No.: US 2021/0339274 A1
(43) Pub. Date: Nov. 4, 2021

(54) TUBULAR HOLDER

(71) Applicant: Michelle Duchnowski, East Meadow, NY (US)

(72) Inventor: Michelle Duchnowski, East Meadow, NY (US)

(21) Appl. No.: 17/373,209

(22) Filed: Jul. 12, 2021

Related U.S. Application Data
(63) Continuation-in-part of application No. 16/055,545, filed on Aug. 6, 2018, now Pat. No. 11,059,060.
Application No. 16/055,545 is a continuation of application No. 15/629,210, filed on Jun. 21, 2017, now Pat. No. 10,040,080.

(51) Int. Cl. B05B 7/14; B05B 7/24; A61K 9/00; B65D 35/00; A45D 40/00; A45D 34/00

(52) U.S. Cl. CPC B05B 7/1431; B05B 7/241; A61K 9/0014; B05B 11/047; A45D 40/00; A45D 34/00; A45D 40/0075; B65D 35/00

(57) ABSTRACT

The present disclosure is directed to a holder. The holder includes a package including a containment portion and a separable portion, wherein the containment portion is separated from the separable portion by perforations, and a sheet within the package, the sheet including a first surface and a second surface, the second surface opposed to the first surface, the first surface operably attached to the separable portion, the sheet configured to extend a distance along an interior of the containment portion, wherein at least a portion of the first surface of the sheet contacts the interior surface of the containment portion, and wherein a liquid composition contacts a second surface of the sheet.

### CROSS REFERENCE TO RELATED APPLICATIONS

The present application is a continuation-in-part a of co-pending application having U.S. Ser. No. 16/055,545, filed on Aug. 6, 2018, which is a continuation of a co-pending application having U.S. Ser. No. 15/629,210, filed on Jun. 21, 2017, now U.S. Pat. No. 10,040,080, the entire contents of which are incorporated herein by reference.

### BACKGROUND OF THE DISCLOSURE

The transport and use of various liquids and/or solids from a user's home, while travelling, is difficult and cumbersome. For example, purchase of a product in a liquid and/or solid form, for example a diaper cream, is typically purchased in a large tub, tube or a rectangular container that is not convenient for travelling with and using outside the home. Further, it is difficult to reliably store a portion of the cream from the original container to another more portable container for later use.

The use of various liquids and/or solids, for example a diaper cream, while travelling can also lead to leakage of the cream and/or drying out of the cream product, rendering the cream product less useful. Also, applying various liquids and/or solids, for example a diaper cream, while a person or child is at another location, such as in a day care setting, or during a diaper change in a home, can be difficult and/or unsanitary, as it requires touch and may result in poor hand hygiene and/or germ transmission.

What is desired is a device and method for storing various liquids and/or solids in a holder that can maintain the liquid and/or solid during storage, make application of the liquid and/or solid easy and be disposed of after use. Embodiments of the present disclosure provide methods that address the above and other issues.

### SUMMARY OF THE DISCLOSURE

The present disclosure is directed to a holder. The holder includes a package including a containment portion and a separable portion, wherein the containment portion is separated from the separable portion by perforations, and a sheet within the package, the sheet including a first surface and a second surface, the second surface opposed to the first surface, the first surface operably attached to the separable portion, the sheet configured to extend a distance along an interior of the containment portion, wherein at least a portion of the first surface of the sheet contacts the interior surface of the containment portion, and wherein a liquid composition contacts a second surface of the sheet, and wherein a liquid composition contacts a second surface of the sheet.

### BRIEF DESCRIPTION OF THE DRAWINGS

The present disclosure will be better understood by reference to the following drawings of which:

FIG. 1A is a perspective view of a main body;

FIG. 1B is a plan view of a film;

FIG. 1C is a plan view of a sheet;

FIG. 1D is a plan view of a sheet;

FIG. 1E is a side view of a sheet;

FIG. 2 is a cross-sectional side view of a holder;

FIG. 3A is a cross-sectional side view of a holder and a user's hand;

FIG. 3B is a cross-sectional side view of a holder, in a non-deformed configuration, and a user's hand;

FIG. 3C is a cross-sectional side view of a holder, in a deformed configuration, and a user's hand; and

FIG. 3D is a plan view of a sheet and a user's hand.

FIG. 3E is a plan view of a sheet and a user's hand.

FIG. 4 is a perspective view of a holder of another embodiment.

FIG. 5 is plan view of the holder of the other embodiment.

FIG. 6 is a perspective view of the holder and two user's hands.

FIG. 7 is a perspective view of the holder and two user's hands.

FIG. 8 is a perspective view of the separable portion of the holder and one of the user's hands.

### DETAILED DESCRIPTION OF THE DISCLOSURE

The present application will now be described in greater detail by referring to the following discussion and drawings that accompany the present application. It is noted that the drawings of the present application are provided for illustrative purposes only and, as such, the drawings are not drawn to scale. It is also noted that like and corresponding elements are referred to by like reference numerals.

In the following description, numerous specific details are set forth, such as particular structures, components, materials, dimensions, processing steps and techniques, in order to provide an understanding of the various embodiments of the present application.

However, it will be appreciated by one of ordinary skill in the art that the various embodiments of the present application may be practiced without these specific details. In other instances, well-known structures or processing steps have not been described in detail in order to avoid obscuring the present application.

It will be understood that when an element as a layer, region or substrate is referred to as being “on” or “over” another element, it can be directly on the other element or intervening elements may also be present. In contrast, when an element is referred to as being “directly on” or “directly over” another element, there are no intervening elements present. It will also be understood that when an element is referred to as being “beneath” or “under” another element, it can be directly beneath or under the other element, or intervening elements may be present. In contrast, when an element is referred to as being “directly beneath” or “directly under” another element, there are no intervening elements present.

In the discussion and claims herein, the term “about” indicates that the value listed may be somewhat altered, as long as the alteration does not result in nonconformance of the process or structure to the illustrated embodiment. For example, for some elements the term “about” can refer to a variation of ±0.1%, for other elements, the term “about” can refer to a variation of ±1% or ±10%, or any point therein.

As used herein, the term “substantially”, or “substantial”, is equally applicable when used in a negative connotation to refer to the complete or near complete lack of an action, characteristic, property, state, structure, item, or result. For example, a surface that is “substantially” flat would either be completely flat, or so nearly flat that the effect would be the same as if it were completely flat.

As used herein terms such as “a”, “an” and “the” are not intended to refer to only a singular entity, but include the general class of which a specific example may be used for illustration.

As used herein, terms defined in the singular are intended to include those terms defined in the plural and vice versa.

Reference herein to any numerical range expressly includes each numerical value (including fractional numbers and whole numbers) encompassed by that range. To illustrate, reference herein to a range of “at least 50” or “at least about 50” includes whole numbers of 50, 51, 52, 53, 54, 55, 56, 57, 58, 59, 60, etc., and fractional numbers 50.1, 50.2 50.3, 50.4, 50.5, 50.6, 50.7, 50.8, 50.9, etc. In a further illustration, reference herein to a range of “less than 50” or “less than about 50” includes whole numbers 49, 48, 47, 46, 45, 44, 43, 42, 41, 40, etc., and fractional numbers 49.9, 49.8, 49.7, 49.6, 49.5, 49.4, 49.3, 49.2, 49.1, 49.0, etc. In yet another illustration, reference herein to a range of from “5 to 10” includes whole numbers of 5, 6, 7, 8, 9, and 10, and fractional numbers 5.1, 5.2, 5.3, 5.4, 5.5, 5.6, 5.7, 5.8, 5.9, etc.

As used herein the term “tubular” refers to an elongate article having an internal chamber running substantially along the length thereof. While it is expected that the tubular products of the present disclosure can have a substantially circular cross-sectional configuration, it is contemplated that other cross-sectional configurations, such as substantially triangular, substantially rectangular, substantially pentagonal (including other geometric shapes with 6, 7 or more sides) and substantially ellipsoidal, may also be used.

As used herein the term “liquid composition” should be understood to encompass one or more of a liquid, a balm, a solution, an aqueous dispersion, a lubricant (such as an automotive lubricant/grease, or a pipe lubricant) a cream, a lotion, a gel, an emulsion, a water-in-oil emulsion, an oil-in-water emulsion, a paste, an oil, a solvent based liquid, an alcohol based liquid, a pH modifying liquid and so forth.

As used herein, the term “non-woven” material should be understood to encompass any non-woven material or fabric. Non-limiting examples of non-woven material/fabric are one or more materials/fabrics based on cellulose fibers, nonwoven papers, textiles including spunbonded webs, dry lay webs, and wet lay webs, biopolymers such as, but not limited to chitosan, poly(N,N-dimethylacrylamide) (PDMA), flexible packaging materials, plastic based materials, polylactic acid, synthetic polymeric fibers such as, but not limited to polyester, polypropylene and combinations thereof. As used herein, the term “cellulose” is used to include any natural and/or synthetic material comprising paper fibers, wood fibers, wood pulp or powder and the like, including cellulosic fibers such as rayon, viscose rayon, multi-limbed viscose, viscose, lyocell, cellulose acetate, cellulose carbamate, and deacetylated cellulose acetate, and regenerated cellulose, e.g., cellophane. Further, any of the non-woven materials disclosed herein can be coated with and/or treated with one or more materials (such as a polymer) to increase or decrease they hydrophobicity. For example, a non-woven cellulose material can be coated with and/or treated with the biopolymer chitosan to increase the hydrophobicity of the non-woven cellulose material.

As used herein, the term “woven” material refers to a fabric containing a structure of fibers, filaments or yarns, which are orderly arranged in an interengaged fashion, woven fabrics typically contain interengaged fibers in a “warp” and “fill” direction. The warp direction corresponds to the length of the fabric while the fill direction corresponds to the width of the fabric. Woven fabrics can be made on a variety of looms including, but not limited to, shuttle looms, Rapier looms, projectile looms, air jet looms and water jet looms.

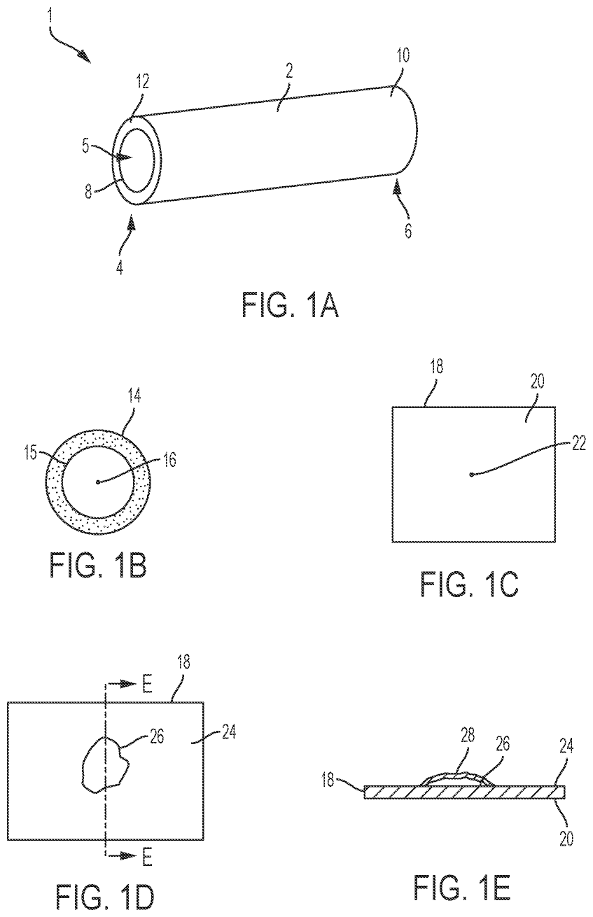

The tubular holder 1 of the present disclosure is illustrated in FIG. 1. The tubular holder 1 includes a tubular main body 2. Tubular main body 2 can be composed of any suitable material, such as but not limited to paper based materials, including cardboard, plastics, metals, ceramic based materials, including glass, carbon based materials, including carbon fibers, and mixtures thereof.

The tubular main body 2 includes a first end 4 and a second end 6, and includes an interior surface 8 and an exterior surface 10. Around the circumference of the first end 4 is a lip 12, which is the transition between the interior surface 8 and the exterior surface 10. An opening 5 extends through the tubular main body 2 from the first end 4 to the second end 6.

The tubular main body 2 can be substantially rigid, or substantially deformable in any direction, with the deformability or the rigidity being adjustable as desired.

In FIG. 1B a film 14 is shown. The film 14 is configured to extend around at least a portion of the circumference of the first end 4 of the tubular main body 2 and also cover the opening 5. The film 14 is shown as substantially circular in FIG. 1B, but, in other embodiments film 14 can be any suitable shape that can extend around at least a portion of the circumference of the first end 4 of the tubular main body 2.

The film 14 can be formed of any suitable woven and/or non-woven material that can maintain a sheet (discussed below) in relation to the tubular main body 2. The sheet is operably attached to the film 14 at least film point 16. Although film point 16 is shown in substantially the center of film 14, the film point 16 can be in any suitable location.

The film 14 can also include an adhesive 15 that is configured to maintain the film 14 in contact with the first end 4. Alternatively or additionally, an adhesive can be included in the vicinity of the lip 12 on the exterior surface 10 of the tubular main body 2.

In FIG. 1C a sheet 18 is shown, with the sheet 18's first surface 20 visible in FIG. 1C. In the figures, sheet 18 is shown as being substantially rectangular for illustrative purposes, but in other embodiments, sheet 18 can be any suitable shape, such as substantially circular, substantially elliptical, substantially triangular, a shape with an erratic outline, or a shape with five or more sides.

Sheet 18 also includes a second surface opposed to the first surface, which is shown in further figures below. On the first surface 20 a sheet point 22 is included, which is configured to be operably attached to the film point 16 in any suitable way, such as by an adhesive, a heat and/or sonic seal, and/or by a mechanical connection such as a staple, a stamping process, sewing through both the sheet 18 and the film 14, or the like. Although sheet point 22 is shown in substantially the center of the sheet 18, the sheet point 22 can be in any suitable location. Also, the point 22 can be larger than a single point, and can cover an area that is about 50% of the area of the opening 5 or about an area that is about 100% of the area of the opening 5.

The opposing surface, second surface 24, of sheet 18 is shown in FIG. 1D. Contacting the second surface 24 is a liquid composition 26. Although the liquid composition 26 is shown as being in substantially the center of the second surface 24, the liquid composition 26 can be in any one or more suitable locations. The sheet 18 can be formed of any suitable woven and/or non-woven material that can maintain the liquid composition 26 in relation to the tubular main body 2. The liquid composition 26 can maintain its position on sheet 18 due to any cohesive and/or adhesive force.

A side cross-sectional view of FIG. 1D is shown in FIG. 1E. In FIG. 1E both the first surface 20 and the second surface 24 can be seen. The liquid composition 26 is shown as contacting the second surface 24, in an embodiment where the sheet is substantially non-absorbent, but, in other embodiments the liquid composition 26 can penetrate a distance into sheet 18 through the second surface 24 towards the first surface 20. Also shown is an optional liquid covering 28 covering at least a portion of the liquid composition 26. Liquid covering 28 can be any substantially impermeable material that can be broken open manually by a user of the tubular holder 1 to allow for egress of the liquid composition 26 contained within and/or on sheet 18. The liquid covering 28 can also surround the liquid composition 26, contacting the second surface 24 around a periphery of the liquid composition 26.

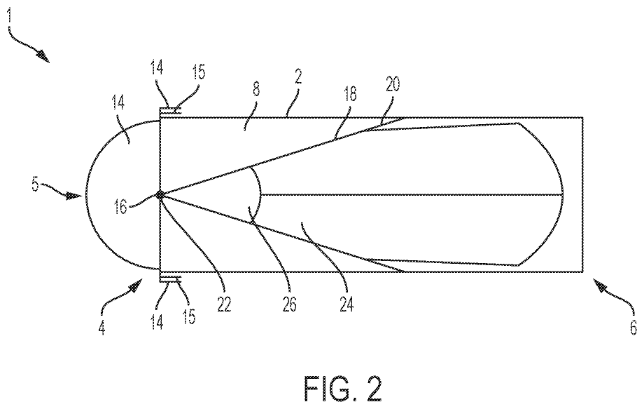

FIG. 2 is a cross sectional view of tubular holder 1, with each of the components of FIGS. 1A-1E shown together.

In this embodiment, the film 14 is extending around a portion of the circumference of the first end 4, covering the opening 5. The adhesive 15 is included in this embodiment, around the circumference of the first end 4, adhering the film 14 to the exterior surface 10 of the tubular main body 2. In other embodiments the adhesive 15 can adhere the film 14 to just the lip 12, or, the adhesive 15 can adhere the film 14 to both the lip 12 and around the circumference of the exterior surface 10 of the first end 4. In other embodiments, the first end 4 can include a rolled edge (or stamped edge) around at least a portion of its exterior periphery, which would provide a larger surface area for connecting the film 14 to the holder 1. Although not shown in this figure, a second film can optionally extend around the second end 6 of the tubular main body 2. In other embodiments, the material of the tubular main body 2 can extend across the second end 6 as a separate element, or as an extension of the exterior surface 10, to cover or substantially cover an opening of the second end 6.

The film point 16 is operably attached to the sheet 18 at the sheet point 22. As can be seen the sheet point 22 is operably attached to the film point 16.

The sheet 18 is shown as extending a distance along an interior of the tubular main body 2 from the first end 4 to the second end 6. In this embodiment a portion of the first surface 20 of the sheet 18 contacts the interior surface 8 of the tubular main body 2. The diameter and circumference of tubular main body 2 can be configured to substantially maintain the sheet 18 in the position of FIG. 2. For example, for a larger surface area sheet 18, a larger diameter tubular main body 2 can be used. As one non-limiting example of the dimensions of these features, a sheet of about four inches in diameter can be used with a tubular main body having about a two inch length and about a ¾ inch diameter. Included in this example the sheet can include a premeasured amount of a liquid composition depending on application needs.

The sheet 18 can be folded or compressed in any suitable way so that it can fit within the tubular main body 2.

Contained by the second surface 24 of the sheet 18, the liquid composition 26 remains substantially stationary within the tubular main body 2 and is substantially maintained in the position of FIG. 2.

The above examples illustrate just a few of the many sizes and shapes each of the components of the tubular holder 1 can be. Other examples can include other shapes and sizes that are capable of performing the same or a similar function.

The methods and devices of the present disclosure will be better understood by reference to the following Examples, which are provided as exemplary of the disclosure and not by way of limitation.

### Example 1

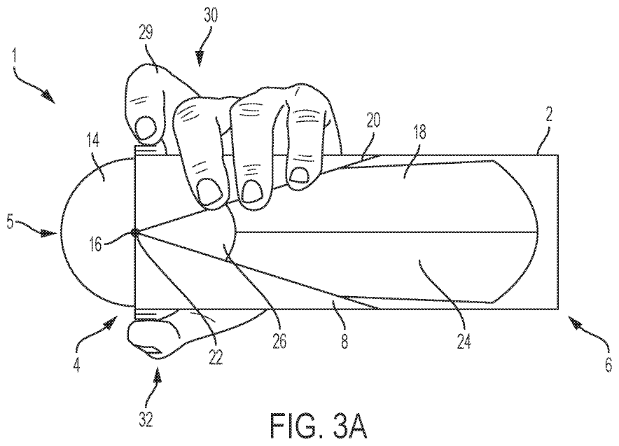

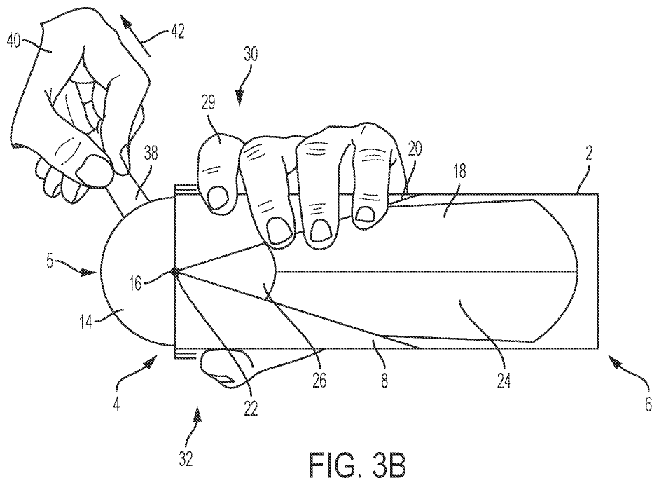

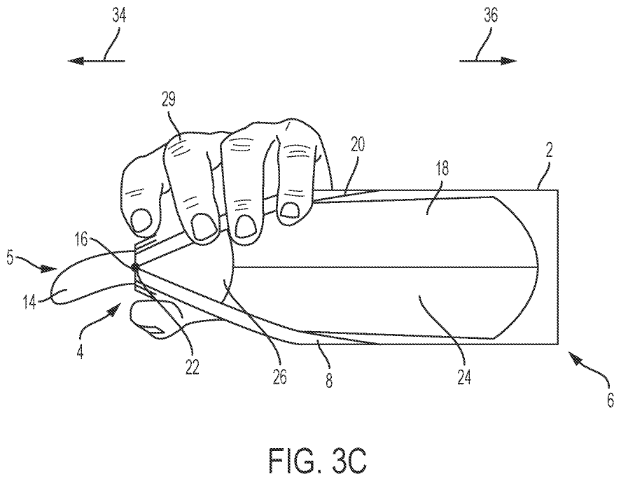

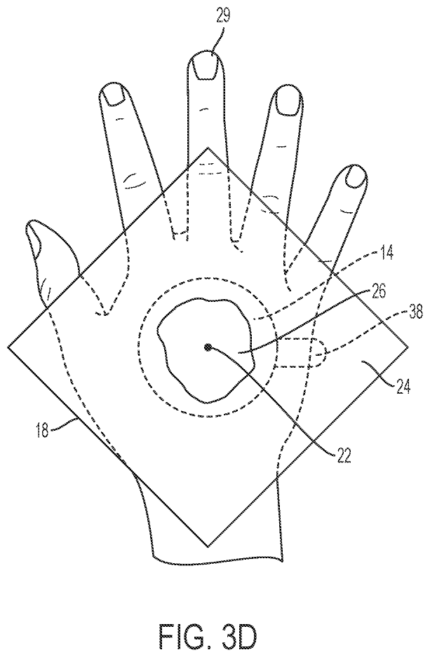

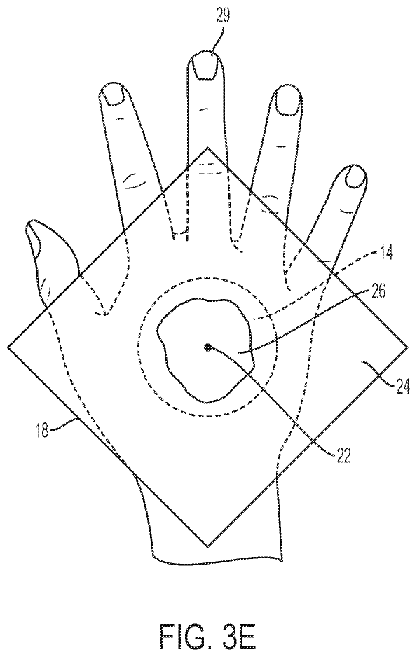

In the present example, a user operates the tubular holder 1 so that liquid composition 26 can be accessed. In FIG. 3A, a user moves their hand 29 so that it is in contact with the tubular main body 2 near the first end 4. In this example, tubular main body 2 is substantially rigid or substantially deformable.

The user then exerts a pressure in substantially the direction of arrows 30 and 32 to substantially maintain the position of the first end 4 of the tubular main body 2. In this embodiment the pressure to the main body is exerted by the user's hand 29, but in other embodiments, any device or implement can be used to apply pressure to the first end 4.

Once pressure is applied, the tubular main body 2 is in the configuration shown in FIG. 3B. With the user's other hand 40 (or another suitable device or implement) a tab 38 and/or the film 14 is grasped and pulled in substantially the direction of arrow 42. Tab 38 is operably connected to the film 14 as a continuous element of the film 14 or a separate piece of material. Tab 38 can be of any suitable shape and size that allows for the tab 38 to be grasped and pulled to remove the film 14 from the tubular main body 2.

Once the tab 38 and/or film 14 are removed from the tubular main body 2, the second surface 24 and liquid composition 26 can be exposed, as shown in FIG. 3D.

In FIG. 3D the user can hold the film 14 and sheet 18, with the first surface 20 contacting the user's hand 29. The second surface 24, along with the liquid composition 26 is then exposed and ready for use and application by the user. In this embodiment the liquid composition 26 is applied to a portion of the user, or another person-such as a child-or any other suitable surface and then all components of the tubular holder 1 are disposed of after the single use.

### Example 2

In the present example, a user operates the tubular holder 1 so that liquid composition 26 can be accessed. In FIG. 3A, a user moves their hand 29 so that it is in contact with the tubular main body 2 near the first end 4. The user may also contact the film 14 with their hand 29. In this embodiment, tubular main body 2 is substantially deformable.

In this example, the user exerts a pressure (from what is shown in FIG. 3A) in substantially the direction of arrows 30 and 32 to deform the first end 4 of the tubular main body 2, as shown in FIG. 3C. In this embodiment the pressure to deform the first end 4 of the main body is exerted by the user's hand 29, but in other embodiments, any device or implement can be used to deform the first end 4. The resulting, deformed tubular main body 2 is shown in FIG. 3C.

Once the tubular main body 2 is in the configuration shown in FIG. 3C, a force is exerted between the film 14 and the tubular main body 2 in substantially the direction of arrow 34 and/or arrow 36. Combined with the deformation force illustrated in FIG. 3A, the force exerted in substantially the direction of arrow 34 and/or arrow 36 causes the adhesive 15 that attaches the film 14 to the tubular main body 2 to detach so that the second surface 24 and liquid composition 26 can be exposed, as shown in FIG. 3E.

In FIG. 3E the user can hold the film 14 and sheet 18, with the first surface 20 contacting the user's hand 29. The second surface 24, along with the liquid composition 26 is then exposed and ready for use by the user. In this embodiment the liquid composition 26 is applied to a portion of the user's skin, or the skin another person-such as a child (or animal such as a cat or dog, for example)—or any other suitable surface and then all components of the tubular holder 1 are disposed of after the single use. Alternatively, or in addition, the liquid composition 26 can be applied to any other suitable element(s), such as any portion of an automobile, a pipe (whereas pipe includes all fittings and/or connections in contact with a length of pipe), a hand tool (such as screw driver), a power tool (such as electric drill), and/or a cooking tool (such as a frying pan).

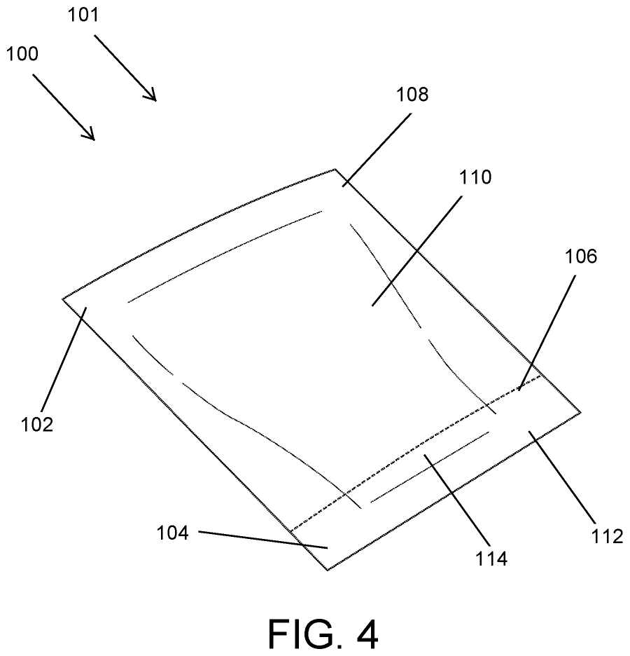

Another embodiment of a holder 100 is shown in FIG. 4, which is a perspective view of the holder 100 in an unopened configuration. In this embodiment, the holder 100 is substantially planar.

The holder 100 includes a package 101 that is composed of a containment portion 102 and a separable portion 104. The containment portion 102 includes a containment sealed portion 108, which substantially extends around the periphery of the containment portion 102 and a containment cavity portion 110 that is substantially surrounded by the containment sealed portion 108. The separable portion 104 includes a separable sealed portion 112 and a separable cavity portion 114.

In this embodiment, the package 101 can be formed of two layers that are joined together to form the containment sealed portion 108 and to form the separable sealed portion 112. These two layers (each of which can contain one or more layers themselves) can be the same material as each other, or each can be different, with these layers being selected from the group comprising one or more metals, one or more plastics, one or more papers or paper products, and combinations thereof, each of which being any suitable thickness. Also, the layers that form the holder 100 can be formed from virgin material, recycled material, or a combination thereof. Further, the package 101 can have varying rigidities, including being rigid, substantially rigid, deformable or substantially deformable, with the deformability or the rigidity being adjustable as desired.

The two layers of the containment sealed portion 108 and the separable sealed portion 112 can be joined to eachother in any suitable way, such as through an adhesive and/or through a melting/heat sealing procedure to form a heat seal. In the melting/heat sealing procedure an additional material may be added between the two layers to act as the melting and sealing material between the two layers. This melting/heat sealing procedure may or may not also include a pressure exerted on or both of the layers.

As used herein, the term “heat seal” refers to any seal of a first region of a film surface to a second region of a film or substrate surface. In some embodiments, the seal can be formed by beating the regions to at least their respective seal initiation temperatures using a heated bar, hot air, infrared radiation, ultrasonic sealing, and/or the like. Alternatively, or in addition to, the heat seal can include an adhesive.

Thus, containment sealed portion 108 and the separable sealed portion 112 comprise a heat seal, which separates the containment sealed portion 108 and the separable sealed portion 112 from the containment cavity portion 110 and the separable cavity portion 114. As will be illustrated and discussed below, a sheet 122 and a liquid composition 126 are stored within the containment cavity portion 110, with a portion of the sheet 122 being secured by the separable portion 104.

A line of perforations 106 can separate the containment portion 102 from the separable portion 104. As seen in FIG. 4, the line of perforations 106 extends across the entire width of the holder 100, but, in other embodiments, there can be one or more portions of perforations 106, and/or one or more notches of cut material can replace some or all of the perforations 106.

As used herein, the term “perforated,” “perforations” and the like terms when used refers to a series of “tabs” spaced apart by “holes,” which connect one or both layers of material of the containment portion 102 to one or both layers of the separable portion 104. “Perforated”, “perforations” and the like can also refer to a weakened area or weakened line that has been mechanically, been subject to a heat/sonic treatment and/or chemically been caused to have a lower tearing force as compared to other portions of the package 101. Tearing along the perforations 106 allows the separable portion 104 and the sheet 118 to be separated from the containment portion 102. Alternative to tearing, or in addition to tearing, the perforations 106 can be disrupted by application of a pressure, such as by an internal gas pressure created by squeezing the containment portion 102.

The perforations 106 can be formed by any suitable method, such as needle perforation (hot or cold), die cutting, laser, water jet and/or hot air pulsing. However, perforations 106 are configured such that the containment cavity portion 110 is completely or substantially isolated from an external environment such that there is little or no transfer of air and/or liquid between an external environment and the interior of the containment cavity portion 110.

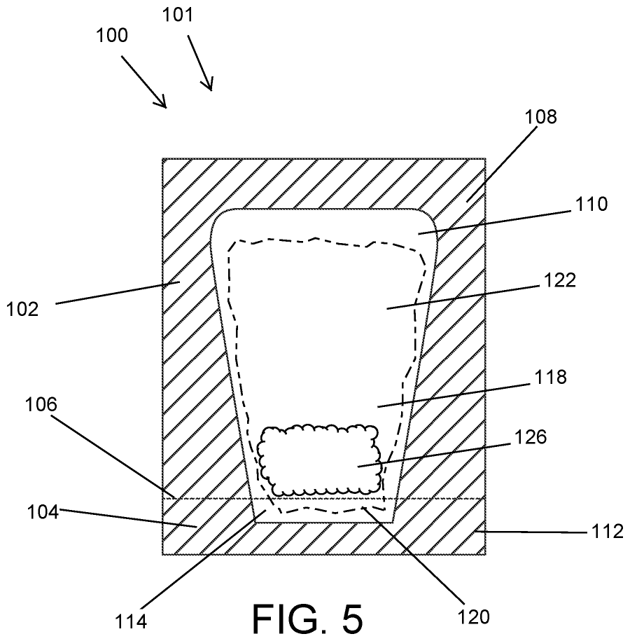

A top view of the holder 100 can be seen in FIG. 5. As can be seen the containment sealed portion 108 and the separable sealed portion 112 form a continuously sealed portion that surrounds both the containment cavity portion 110 and the separable cavity portion 114.

As can be seen in FIG. 5, the sheet 118 contains two areas, the sealed sheet portion 120 and the unfixed sheet portion 122. The sealed sheet portion 120 of sheet 118 is sealed and/or operably attached to the separable portion 104, within the separable cavity portion 114, through a heat seal, or any other suitable connection, such as an adhesive.

The unfixed sheet portion 122 is contained within the containment cavity portion 110, but not attached to the containment portion 102. Specifically, an outer surface of the unfixed sheet portion 122 is contained within the containment cavity portion 110, is not attached to the containment portion 102, but does contact one or more points within the containment cavity portion 110. Contained substantially within an inner surface of the unfixed sheet portion 122 is a volume of a liquid composition 126.

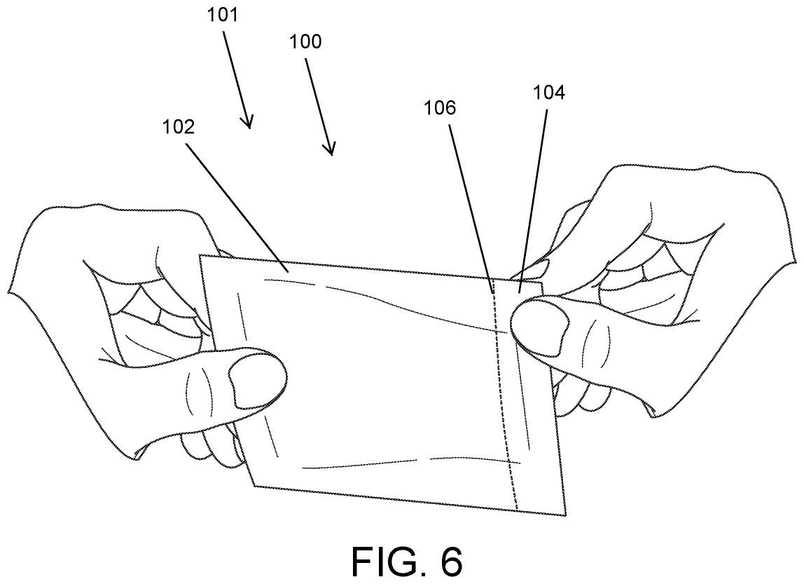

Use and operability of the holder 100 is discussed in reference to FIGS. 6-8.

As can be seen in FIG. 6, a user can grasp the separable portion 104 and/or the containment portion 102 and exert sufficient force on perforations 106 such that the separable portion 104 begins to separate from the containment portion 102. The force exerted on the separable portion 104 and/or the containment portion 102 in any suitable direction and can also include a twisting or rotating component.

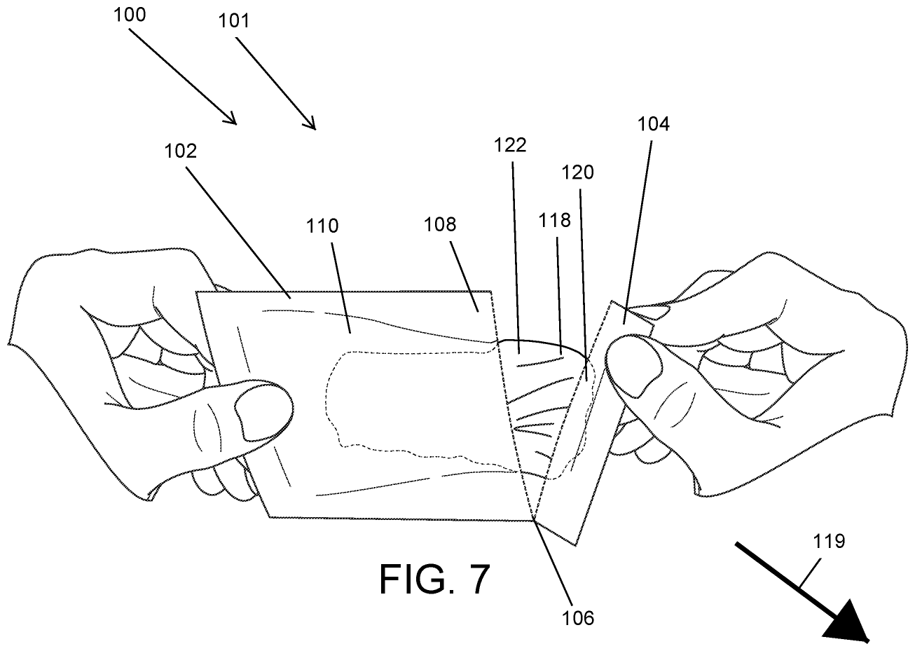

After a sufficient amount of force is applied, the separable portion 104 separates, wholly or partially, from the containment portion 102, as shown in FIG. 7, which illustrates a nearly whole separation of the separable portion 104 from the containment portion 102.

As can be seen from FIG. 7, and in this example, a force in the direction of arrow 119 has been exerted on the separable portion 104 such that only a small amount of perforations 106 remain connecting the separable portion 104 to the containment portion 102. As the user continues to exert force in the direction of arrow 119, the sheet 118 is withdrawn from the containment cavity portion 110 due to a portion of the sheet 118 being sealed and/or operably attached to the separable portion 104 at the sealed sheet portion 120. In the configuration of FIG. 7, the liquid composition 126 would not be visible since an outer surface of the sheet 118 is visible by the user.

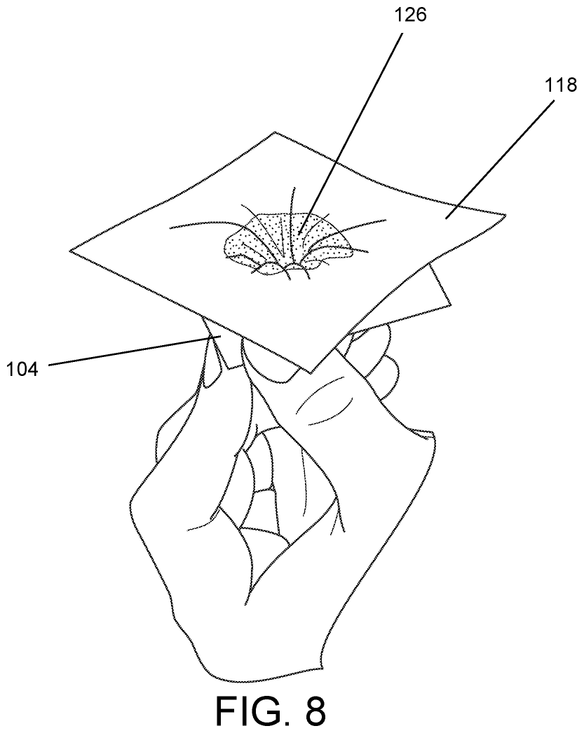

As the user continues to apply force in the direction of arrow 119, the perforations 106 can all be torn such that a user is holding the separable portion 104, with the attached sheet 118 and the attached sheet 118's stored liquid composition, which is shown in FIG. 8. In FIG. 8, the user has separated the separable portion 104 from the containment portion 102, and has spread out the sheet 118 so that the inner surface is shown, with the inner surface of the sheet 118 supporting the liquid composition 126.

As shown in FIG. 8, the liquid composition 126 is then exposed and ready for use and application by the user. In this embodiment the liquid composition 126 is applied to a portion of the skin of the user, or another person's skin-such as a child (or animal such as a cat or dog, for example)—or any other suitable surface and then all components of the holder 100 can be disposed of after the single use.

Alternatively, or in addition, the liquid composition 26 can be applied to any other element(s), such as any portion of an automobile, a pipe (whereas pipe includes all fittings and/or connections in contact with a length of pipe), a hand tool (such as screw driver), a power tool (such as electric drill), and/or a cooking tool (such as a frying pan).

In FIG. 8, sheet 118 is shown as being substantially rectangular for illustrative purposes, but in other embodiments, sheet 118 can be any suitable shape, such as substantially circular, substantially elliptical, substantially triangular, a shape with an erratic outline, or a shape with five or more sides.

Although the liquid composition 126 is shown as being in substantially the center of the sheet 118 in FIG. 8 as well as in FIG. 5, the liquid composition 126 can be in any one or more suitable locations. Also, the sheet 118 can be formed of any suitable woven and/or non-woven material that can maintain the liquid composition 126 in relation to the sheet 118. The liquid composition 126 can maintain its position on sheet 118 due to any cohesive and/or adhesive force.

Although not shown, an optional liquid covering can cover at least a portion of the liquid composition 126. Liquid covering can be any substantially impermeable material that can be broken open manually by a user of the holder 100 to allow for egress of the liquid composition 126. The liquid covering can also surround the liquid composition 126, contacting the second surface of the sheet 118 around a periphery of the liquid composition 126. The described embodiments and examples of the present disclosure are intended to be illustrative rather than restrictive, and are not intended to represent every embodiment or example of the present disclosure. While the fundamental novel features of the disclosure as applied to various specific embodiments thereof have been shown, described and pointed out, it will also be understood that various omissions, substitutions and changes in the form and details of the devices illustrated and in their operation, may be made by those skilled in the art without departing from the spirit of the disclosure. For example, it is expressly intended that all combinations of those elements and/or method steps which perform substantially the same function in substantially the same way to achieve the same results are within the scope of the disclosure. Moreover, it should be recognized that structures and/or elements and/or method steps shown and/or described in connection with any disclosed form or embodiment of the disclosure may be incorporated in any other disclosed or described or suggested form or embodiment as a general matter of design choice. Further, various modifications and variations can be made without departing from the spirit or scope of the disclosure as set forth in the following claims both literally and in equivalents recognized in law.

1. A holder, the holder comprising:
a package comprising a containment portion and a separable portion, wherein the containment portion is separated from the separable portion by perforations and/or a weakened line;
a sheet within the package, the sheet comprising a first surface and a second surface, the second surface opposed to the first surface, the first surface operably attached to the separable portion, the sheet configured to extend a distance along an interior of the containment portion, wherein at least a portion of the first surface of the sheet contacts the interior surface of the containment portion, and wherein a liquid composition contacts a second surface of the sheet.

2. The holder of claim 1, wherein the sheet is formed of a woven material, a non-woven-material, and combinations thereof.

3. The holder of claim 2, wherein the sheet is coated and/or treated with one or more polymers.

4. The holder of claim 1, wherein the package is substantially deformable.

5. The holder of claim 1, wherein the liquid composition is selected from the group consisting of a balm, a lubricant, a cream, a lotion, a gel and a paste.

6. A method of applying a liquid composition comprising:
removing a separable portion from a containment portion of a package, wherein the containment portion is separated from the separable portion by perforations and/or a weakened line, and wherein a first surface of a sheet is operably attached to the separable portion, wherein the sheet comprising the first surface and a second surface, the second surface opposed to the first surface, the sheet configured to extend a distance along an interior of the containment portion, wherein at least a portion of the first surface of the sheet contacts the interior surface of the containment portion, and wherein a liquid composition contacts a second surface of the sheet;
exposing the liquid composition from the sheet; and
applying the liquid composition to a surface.

7. The method of claim 6, wherein the surface is a surface selected from the group consisting of a human's skin, an automobile, a pipe, and a tool.

8. The holder of claim 6, wherein the liquid composition is selected from the group consisting of a balm, a lubricant, a cream, a lotion, a gel and a paste.
